(19) United States
(12) Patent Application Publication
(10) Pub. No.: US 2026/0215869 A1
(43) Pub. Date: Jul. 30, 2026

(54) CONTROL OF A SURGICAL ROBOT ARM

(71) Applicant: CMR Surgical Limited, Cambridge (GB)

(72) Inventors: David Christopher MOORE, Cambridge (GB); Gregory Vincent STUART, Cambridge (GB); Edward James Wildin TUCKER, Cambridge (GB); Johnathan Man Chiu HUNG, Cambridge (GB); John Daniel TILL, Cambridge (GB)

(73) Assignee: CMR Surgical Limited, Cambridge (GB)

(21) Appl. No.: 19/141,589

(22) PCT Filed: Dec. 18, 2023
(86) PCT No.: PCT/GB2023/053285

§ 371 Date: Jun. 20, 2025

(30) Foreign Application Priority Data
Dec. 20, 2022 (GB) 2219316.3

(51) Int. Cl. A61B 34/37; A61B 34/30; A61B 90/00

(52) U.S. Cl. CPC A61B 34/37; A61B 90/03; A61B 2034/306; A61B 2090/064

(57) ABSTRACT

A control unit for controlling motion of a surgical robot arm, the control unit being configured to receive an input signal from a surgeon input device for controlling the surgical robot arm and send an output signal to the surgical robot arm to cause motion of the surgical robot arm, the control unit being further configured to: detect a clash in respect of the surgical robot arm; in response to detecting the clash, place a limitation on the motion of the surgical robot arm; receive an input signal from the surgeon input device for controlling the surgical robot arm and send an output signal to the surgical robot arm so as to control motion of the surgical robot arm in accordance with the limitation.

### BACKGROUND

It is known to use robots for assisting and performing surgery. FIG. 1 shows a surgical robot arm 102 which comprises a base 101 and is attached to an implement 103. The base supports the rest of the surgical robot arm, and is itself attached rigidly to, for example, the operating theatre floor, the operating theatre ceiling or a trolley. The arm further comprises a plurality of arm segments 105 connected by a plurality of driven joints 104 extending between the base 101 and a terminal arm segment 105t. The surgical robot arm 102 is articulated by the multiple driven joints 104 between the arm segments which are used to position the implement 103 at a desired location and orientation relative to a patient. The driven joints 104 include the elbow 104e and the wrist 104w. As seen in FIG. 1, a roll joint 104r separates the terminal arm segment 105t from the wrist joint 104w. The wrist joint 104w is therefore the most distal joint in the arm that is not a roll joint.

The implement 103 is attached to the distal end of the terminal arm segment 105t of the robot arm. During an operation, the implement can penetrate the body of a patient at a port so as to access a surgical site. In the example seen in FIG. 1, the implement is a surgical instrument having a pair of jaws. Other surgical instruments may include a knife, a cauterizer, a hook, an electrosurgical instrument and a pair of scissors. In other examples, the implement may be an endoscope.

FIG. 1 illustrates that the surgical robot arm 102 forms part of a system 100 also including a surgeon command interface 106 (which may be referred to as a “surgeon console”) and a control unit 107. The control unit comprises a processor 108 and a memory 109. The memory 109 of the control unit 107 stores in a non-transient way software that is executable by the processor 108 to cause the arm 102 to move as desired. The control unit is coupled to actuators (not shown) in the surgical robot arm 102. The actuators are coupled to the joints 104 in the robot arm such that driving the actuators causes the angles of the joints to change, causing the arm to move. The control unit 107 can therefore effect motion of the robot arm 102 in accordance with outputs generated by execution of the software.

The control unit 107 may, or may not, be remote from the arm 102.

The processor 108 of the control unit 107 can execute the software to drive the arm in dependence on inputs from a surgeon command interface 106. The surgeon command interface 106 may comprise one or more surgeon input devices whereby a user (a surgeon) can request motion of the implement in a desired way. The control unit 107 is configured to receive an input signal from the surgeon input device 106. The input devices could, for example, be manually operable mechanical input devices such as hand controllers or joysticks, or contactless input devices such as optical gesture sensors. In the following description, the surgeon input device is a set of hand controllers. The surgeon can move the hand controllers so as to request the desired motion of the implement. The software stored in memory 109 of the control unit 107 is configured to respond to input signals received from the surgeon input device and the processor 108 is configured to execute the software to send an output signal to the actuators of the surgical robot arm. The control unit is therefore configured to send an output signal to cause the joints of the arm to move in accordance with the input signal received from the surgeon input device to achieve the requested motion of the implement. In other words, the input signal constitutes a request for a particular position and orientation of the implement and the control unit is configured to translate the request into an output signal which causes joints in the arm to move to the angles necessary to achieve the requested implement position and orientation.

When the surgical robot arm is in a compliant mode, the processor can also execute the software to drive the arm in dependence on an external force acting on the arm 102. The control unit 107 is configured to receive an input signal as a result of the external force acting on the arm, the input signal being indicative of the force, position and direction of the external force acting on the arm. For example, the control unit 107 is able to cause the arm to move in response to a member of the bedside team pushing on a part of the arm, thereby giving the impression that the member of the bedside team is physically moving the arm.

The system 100 shown in FIG. 1 may be part of a larger system including one or more further surgical robot arms. Each surgical robot arm may include its own base. In such a system, the control unit 107 is configured to receive an input signal from the surgeon input device with respect of each of the surgical robot arms. The software stored in memory 109 of the control unit is configured to respond to those inputs and the processor 108 is configured to execute the software to send an output signal to the actuators of any of the one or more further surgical robot arms to cause the joints of the respective arm to move in accordance with the input signal from the surgeon input device. The surgeon is thereby able to control the motion of each of the surgical robot arms using the surgeon input device. As well as the previously described control unit 107 and surgeon command interface 106, a larger surgical system may include the surgical robot arm 102 illustrated in FIG. 1 which is connected to a surgical instrument (an instrument arm) and may include a second robot arm which is connected to an endoscope (an endoscope arm). The surgeon command interface 106 includes a display on which the surgeon is able to view the surgical site by means of a signal received from the endoscope.

It is common, particularly in systems which include multiple surgical robot arms, that clashes occur. For example, a surgical robot arm may experience a clash while a surgical procedure is being carried out. As will be explained in more detail below, the term clash is used herein to refer to a circumstance in which one or more conditions are met indicating that normal operation of the surgical robot arm (e.g. for controlling the motion and/or position of the surgical robot arm or attached implement) might not be possible. The result of a clash occurring is often that the surgical robot arm can no longer be driven as desired. For example, the clash may cause errors to be introduced between the position of the implement requested by the surgeon using the surgeon input device and the achieved position of the implement. Thus after a clash has occurred, the surgeon may no longer be able to control the surgical robot arm to position the implement at the desired location. A clash may be caused by a collision between a surgical robot arm and another object, for example a clash may be caused by a collision between two surgical robot arms. A clash may be caused by a collision between an implement and another object, such as another implement or a surgical robot arm. A clash may not be caused by a collision.

In response to a clash, the surgeon input device may be disengaged from the surgical robot arm, such that movement of the surgeon input device does not affect the position of the surgical robot arm. The clash can then be resolved while the surgeon input device is disengaged, e.g. a member of the bedside team may physically move the surgical robot arm to stop it colliding with another object. When the clash has been resolved, the surgeon input device may be reengaged with the surgical robot arm, such that movement of the surgeon input device can control the position of the surgical robot arm again.

The present application concerns improvements in responding to clashes in respect of a surgical robot arm.

### SUMMARY

According to a first embodiment there is provided a control unit for controlling motion of a surgical robot arm, the control unit being configured to receive an input signal from a surgeon input device for controlling the surgical robot arm and send an output signal to the surgical robot arm to cause motion of the surgical robot arm, the control unit being further configured to: detect a clash in respect of the surgical robot arm; in response to detecting the clash, place a limitation on the motion of the surgical robot arm; receive an input signal from the surgeon input device for controlling the surgical robot arm and send an output signal to the surgical robot arm so as to control motion of the surgical robot arm in accordance with the limitation.

The surgical robot arm may comprise a plurality of arm segments connected by a plurality of driven joints extending between a base and a terminal arm segment and the terminal segment of the surgical robot arm may be configured to connect to an implement.

The clash that is detected in respect of the surgical robot arm may be a clash on the surgical robot arm or a clash on an implement connected to the surgical robot arm.

The implement may be a surgical instrument or an endoscope.

Detecting a clash in respect of the surgical robot arm may comprise: one or both of determining a torque value representing the torque at each of the plurality of joints, determining a parameter based on the determined torque value and determining that the parameter exceeds a first torque threshold; and determining a position error of a part of the surgical robot arm or part of the implement connected to the surgical robot arm, and determining that the determined position error exceeds a first position error threshold.

The control unit may be further configured to determine whether the detected clash in respect of the surgical robot arm is a severe clash, wherein determining whether the detected clash is a severe clash comprises determining that the determined parameter exceeds a second torque threshold and/or that the determined position error exceeds a second position error threshold, wherein the second torque threshold is greater than the first torque threshold and the second position error threshold is greater than the first position error threshold.

The control unit may be configured to, in response to determining that the detected clash is a severe clash, place a first limitation on the motion of the surgical robot arm for a first duration, wherein the first limitation is a complete restriction of the speed of motion of terminal arm segment, wherein the control unit is further configured to place a second limitation on the motion of the surgical robot arm for a second duration after the first duration, wherein the second limitation is a restriction of the speed of motion of the terminal arm segment to a maximum value, the maximum value being greater than zero.

The second duration may be 2 seconds.

The limitation may comprise one or both of a restriction of the speed of motion of the surgical robot arm and a restriction of the direction of motion of the surgical robot arm.

The restriction of the speed of motion of the surgical robot arm may comprise a complete restriction of the speed of motion of the terminal arm segment so as to suspend motion of the terminal arm segment.

The control unit may be configured to place the complete restriction on the speed of motion of the terminal arm segment for 0.75 seconds.

The restriction of the speed of motion of the surgical robot arm may be a restriction of the speed of motion of the terminal arm segment to a maximum value, the maximum value being greater than zero.

The control unit may further be configured to determine whether the clash affects the motion of a particular joint of the surgical robot arm or the motion of the implement, wherein determining whether the clash affects the motion of the particular joint of the surgical robot arm or the motion of the implement comprises one or both of: determining a force at the particular joint, and determining that the determined force exceeds a force threshold; and determining a position error of the distal end of the implement, and determining that the determined position error exceeds an implement position error threshold.

The control unit may be configured to, in response to determining that the clash affects the motion of the particular joint of the surgical robot arm or the motion of the implement, determine a vector associated with the clash.

The vector associated with the clash may have a direction and a tip, the direction of the vector representing the direction of the clash incident on the surgical robot arm or implement, and the tip of the vector representing a point on the surgical robot arm or implement at which the clash is incident.

The point on the surgical robot arm or implement at which the clash is incident may be a point on the particular joint, the terminal arm segment, an arm segment connected to the particular joint, or the implement.

The limitation may be a restriction of the direction of motion of the point on the surgical robot arm or implement to motion in a direction within a virtual cone and away from an apex of the virtual cone, the apex of the virtual cone being located at the point on the surgical robot arm or implement at which the clash is incident and the central axis of the virtual cone being colinear with the vector associated with the clash.

The apex angle of the virtual cone may be within a range from 40 to 100 degrees.

The plurality of driven joints may comprise a wrist joint which is the most distal of the driven joints that is not a roll joint; and the particular joint of the plurality of driven joints is the wrist joint.

The force threshold for the wrist joint may be 35 N and the implement position error threshold may be 15 mm.

According to a second embodiment there is provided a surgical system comprising a control unit as previously described and a surgical robot arm.

The surgical robot arm may comprise a terminal arm segment connected to an endoscope.

The system may comprise one or more further surgical robot arms and the control unit is configured to: for each of the one or more further surgical robot arms, receive an input signal from the surgeon input device for controlling the surgical robot arm and send an output signal to the surgical robot arm to cause motion of the surgical robot arm; and in response to detecting the clash, place a limitation on the motion of each of the one or more further surgical robot arms, wherein the limitation comprises a complete restriction of the speed of motion of the terminal arm segment of that one of the one or more further surgical robot arms so as to suspend motion of the terminal arm segment of that one of the one or more further surgical robot arms.

According to a third embodiment, there is provided a method for controlling motion of a surgical robot arm, the method comprising: detecting a clash in respect of the surgical robot arm; in response to detecting the clash, placing a limitation on the motion of the robot arm; receiving an input signal from a surgeon input device for controlling the surgical robot arm and sending an output signal to the surgical robot arm so as to control motion of the surgical robot arm in accordance with the limitation.

According to a fourth embodiment there is provided a non-transitory computer readable storage medium having stored thereon computer readable instructions that, when executed at a computer system, cause the computer system to perform the previously described method.

### BRIEF DESCRIPTION OF THE FIGURES

FIG. 1 illustrates a surgical system including a surgical robot arm and a control unit.

FIG. 2 illustrates a general method performed by the control unit to control motion of a surgical robot arm in response to a clash.

FIG. 3 illustrates the first stage of a specific method performed by the control unit when a clash is detected.

FIG. 4 illustrates the stage of the specific method performed by the control unit when the clash is determined to be a severe clash.

FIG. 5 illustrates the stage of the specific method performed by the control unit when the clash is determined not to be a severe clash.

FIG. 6 illustrates the stage of the specific method performed by the control unit when the clash is determined not to affect the motion of the wrist of the surgical robot arm or implement attached to the surgical robot arm.

FIG. 7 illustrates the stage of the specific method performed by the control unit when the clash is determined to affect the motion of the wrist of the surgical robot arm or implement attached to the surgical robot arm.

FIG. 8 illustrates the distal end of the surgical robot arm and virtual quantities determined as part of the method of FIG. 7.

### DETAILED DESCRIPTION

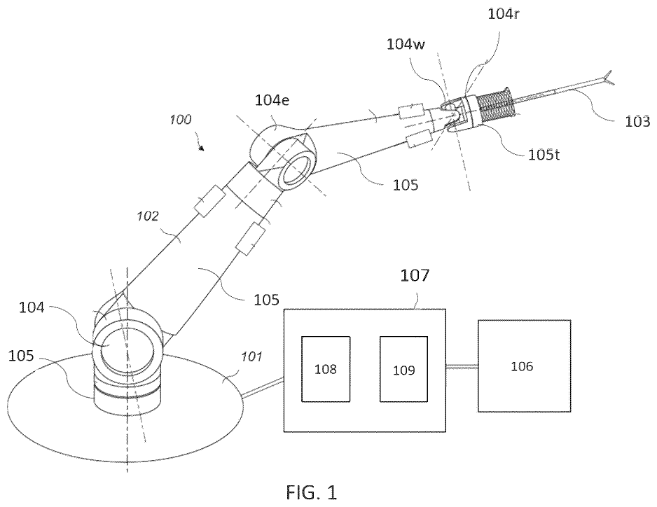

As explained above with respect to FIG. 1, when the surgical robot arm is in a compliant mode, the control unit 106 is configured to drive surgical robot arm 102 in response to input signals received from a surgeon input device at surgeon command interface 106 and in response to input signals received as a result of an external force acting on a part of the arm (for example due to a member of the bedside team pushing on it).

As previously mentioned, the result of a clash occurring on the robot arm may be that the surgical robot arm can no longer be driven as desired. For example, the clash may cause errors to be introduced between the position of the implement requested by the surgeon using the surgeon input device and the achieved position of the implement. In cases where the clash is caused by a collision, position errors may occur due to an external object acting as an obstacle to the requested motion of the robot arm. Therefore, if a clash occurs during a surgical procedure, it is necessary to resolve the clash, for example by repositioning the arm so as to avoid further collision, so that usual control of the robot arm can be resumed.

Furthermore, allowing the surgeon to continue to control motion of the surgical robot arm after a clash has occurred and before it has been resolved can be dangerous as the desired motion of the implement requested by the surgeon does not always translate effectively into the implement's achieved position. It is therefore desirable to control the motion of the surgical robot arm after a clash has occurred and before the clash has been resolved in a manner which improves the safety of the surgical procedure.

As mentioned in the background section above, one approach to improving the safety of the surgical procedure is to detect that a clash has occurred and in response to detecting that a clash has occurred, disconnect the surgeon input device from the control unit such that the control unit no longer drives the surgical robot arm in response to input signals received form the surgeon input device. In other words, the surgeon is disengaged from the robot arm and is no longer able to dictate motion of the attached implement. When the surgeon is disengaged from the surgical robot arm, they may be able to move the hand controllers, for example to increase their workspace or navigate a menu, but such movement will not result in any change to the position of the surgical robot arm. Adopting such an approach may mean that the control unit is only able to drive the surgical robot arm in response to input signals received as a result of an external force acting on the arm, but not in response to input signals received from a surgeon input device. In such a case, where a clash is detected, since the surgeon is no longer able to influence motion of the robot arm, the clash can only be resolved by imparting an external force on the arm. According to some examples, the arm may comprise a control on the arm which may be actuatable by a member of the bedside team to move the arm. In other words, a member of the bedside team is required to reposition the arm to resolve the clash. Although adopting this approach can improve the safety of the surgical procedure, the approach can result in significant delays to the surgical procedure due to the frequent disengagement of the surgeon from the robot arm. Each time the surgeon is disengaged, they are required to reengage the robot arm by actuating a control on the surgeon input device. As well causing delays to the procedure, it has been found that surgeons find frequent disengagement frustrating, thereby decreasing their satisfaction with the surgical system. Furthermore, it can be the case that the surgeon is better placed than the beside team to know how to reposition the robot arm so as to resolve the clash.

An alternative approach, described herein, has therefore been developed which improves the safety of the surgical procedure while minimising delays during surgical procedures. This approach does not require the surgeon to be entirely disengaged from the robot arm, but instead places a limitation on the motion of the robot arm controllable by the surgeon input device. In other words, after a clash is detected, the control unit continues to receive an input signal from the surgeon input device but places a limitation on the motion of the surgical robot arm and therefore responds to receiving the input signal from the surgeon input device by issuing an output signal to the robot arm which causes motion of the robot arm in accordance with the limitation. Broadly, the motion controllable by the surgeon input device is limited to motion which enables a detected clash to be resolved safely. Clashes may therefore be resolved based on actions taken by both the bedside team and the surgeon. In other words, the surgeon need not rely on members of the bedside team to resolve a clash and thus has increased control over the surgical system. This has been found to enable some clashes to be resolved more quickly and has resulted in improved surgeon satisfaction with the surgical system.

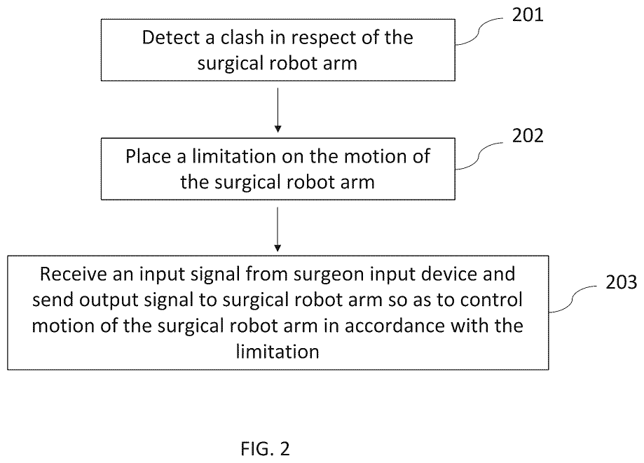

FIG. 2 illustrates a method performed by the control unit 107 to control the motion of the surgical robot arm 102 after a clash has occurred. The first step 201 of the method performed by the control unit is to detect a clash in respect of the surgical robot arm.

Detecting a clash in respect of the surgical robot arm is used herein to mean detecting that a part of the surgical robot arm or a part of the attached implement meets one or more conditions relating to the motion and/or position of the surgical robot arm or implement.

For example, detecting a clash in respect of the surgical robot arm may comprise determining a value for a variable relating to the motion and or position of the robot arm or implement and comparing that value to a threshold value for the variable.

Detecting a clash in respect of the surgical robot arm may comprise determining a torque value representing the torque at each of the plurality of joints, determining a parameter based on the determined torque value and determining that the parameter exceeds a first torque threshold.

The surgical robot arm 102 seen in FIG. 1 may further include one or more torque sensors (not shown) at each of the driven joints 104. Determining a torque value representing the torque at each of the plurality of joints may therefore comprise receiving measurements from one or more of the torque sensors. The torque value may comprise a vector containing the magnitude of the torque measured at each of the driven joints. The magnitude of the torque measured at each of the driven joints may be adjusted so as to remove one or more of torques resulting from gravity due to the mass of the robot arm, torques resulting from internal gear train stretching and/or reaction torques resulting from the attached implement interacting with the port and surgical site. The parameter based on the determined torque value may therefore be a compensated adjusted torque for one or more of the plurality of joints. The torque sensors may be configured to determine the torque at each joint at regular time intervals. Determining a parameter based on the determined torque value may therefore comprise determining a difference between torque values determined at different time intervals. The parameter based on the determined torque value may be a difference between torque values determined at consecutive time intervals.

The parameter may therefore represent the change in torque at one or more joints between consecutive torque measurements. The parameter based on the determined torque value may be a difference between gravity compensated adjusted torques determined at consecutive time intervals.

Detecting a clash in respect of the surgical robot arm may comprise determining a position error of a part of the surgical robot arm or part of the implement and determining that the determined position error exceeds a first position error threshold.

The surgical robot arm 102 seen in FIG. 1 may further include one or more position sensors (not shown) at one or more arm segment, joint or implement. Determining a position error of the part of the surgical robot arm or implement may therefore comprise receiving measurements from one or more of the position sensors.

The part of the surgical robot arm at which the position error is determined may be a joint of the plurality of joints. The position error may be an error of the joint angle of one or more of the plurality of joints. An error of the joint angle may be the difference between the joint angle derived from the input position of the actuator used to drive the joint and the actual output joint angle. An error of the joint angle may be the difference between the joint angle requested (as a result of the signal received from the surgeon input device) and the actual output joint angle. The first position error threshold may thus comprise a joint angle error threshold corresponding to one or more of the joints of the plurality of joints.

The part of the implement at which the position error is determined may be the tip of the implement. The position error may be the distance between the requested position of the tip of the implement (as a result of the signal received from the surgeon input device) and the actual position of the tip of the implement. The actual position of the tip of the implement may be determined by one or more of the position sensors present on the arm

The first position error threshold may be a distance of 20 mm. The part of the implement at which the position error is determined may be the longitudinal axis of the implement. The position error may be the distance between the longitudinal axis of the implement and a selected point in the surgical site about which the implement is controlled to move. The position error may be the distance between the longitudinal axis of the implement and a virtual pivot point in the surgical site. The first position error threshold may be a distance of 15 mm.

The surgical robot arm 102 is illustrated in FIG. 1 to be in an unfolded position in which it is able to perform surgical procedures. When the arm is not in use, it can be folded into a more compact configuration for storage purposes. The one or more conditions relating to the motion and/or position of the surgical robot arm used to detect a clash may relate to the motion and/or position of the arm when it is either in the compact folded configuration or while the arm is being folded or unfolded.

According to one example, in which the surgical robot arm has 8 joints and the joints are numbered in order starting from the base, detecting a clash in respect of the surgical robot arm may comprise determining that any one or more of the following conditions (1) to (7) is true.
1. That, for any of joints 1 to 8, the magnitude of the gravity compensated adjusted torque about that joint exceeds a threshold value for the joint. The following thresholds may be used.

TABLE 1

| Joint | Threshold (Nm) |
|---|---|
| 1 | 30 |
| 2 | 30 |
| 3 | 40 |
| 4 | 20 |
| 5 | 5 |
| 6 | 5 |
| 7 | 5 |
| 8 | 2 |

2. That, for any two joints of the first seven joints in the plurality of joints, the difference between the gravity compensated adjusted torque value at consecutive time intervals exceeds the threshold values for those joints. The following thresholds may be used.

TABLE 2

| Joint | Threshold (Nm) |
|---|---|
| 1 | 0.1 |
| 2 | 0.2 |
| 3 | 0.2 |
| 4 | 0.2 |
| 5 | 0.05 |
| 6 | 0.02 |
| 7 | 0.02 |

3. That, the difference between the joint angle derived from the input actuator position and the actual output joint angle, for one or both of joints 6 and 7, exceeds a threshold value for the joint. The following thresholds may be used.

TABLE 3

| Joint | Threshold (rad) |
|---|---|
| 6 | 0.02 |
| 7 | 0.02 |

4. That, for one or more joints of joints 3 to 8, the difference between the requested joint angle and the actual output joint angle exceeds a threshold value for the joint. The following thresholds may be used.

TABLE 4

| Joint | Threshold (rad) |
|---|---|
| 3 | 0.01 |
| 4 | 0.005 |
| 5 | 0.02 |
| 6 | 0.02 |
| 7 | 0.02 |
| 8 | 0.06 |

5. That, the distance between the requested position of the tip of the implement and the actual position of the tip of the implement exceeds 15 mm.
6. That, the distance between the longitudinal axis of the implement and a virtual pivot point in the surgical site exceeds 15 mm.
7. That, while the arm is either in the compact folded configuration or while the arm is being folded or unfolded and the wrist joint 104w of the arm is less than 10 cm from its final folded position, either:
   the difference between the requested joint angle of joint 6 and the actual output joint angle of joint 6 exceeds 0.01 radians; or
   the magnitude of the gravity compensated adjusted torque about joint 6 exceeds 1.5 Nm.

As will be explained in more detail below, the control unit may continue to check for clashes at regular time intervals. This enables the control unit to determine when a detected clash has been resolved. Determining that a clash has been resolved may require detecting that no parts of the surgical robot arm or parts of the attached implement meet the one or more conditions relating to the motion and/or position of the surgical robot arm or implement described above.

After the clash has been detected, the control unit may send a signal to cause an audible alert to be issued by the surgical system to alert the surgeon and members of the bedside team that a clash has been detected. The control unit may send a signal to the surgeon command interface so that icons are shown on the display of the interface which indicate that a clash has been detected.

As seen in FIG. 2, after the clash has been detected, at step 202, the control unit places a limitation on the motion of the surgical robot arm. As will be described in more detail below, the limitation may be a restriction of the speed of motion of the surgical robot arm.

The limitation may be a restriction of the direction of motion of the surgical robot arm. The limitation may be a restriction of both of the speed and direction of motion of the surgical robot arm. The restriction of the speed of motion of the surgical robot arm may be a complete restriction of a part of the surgical robot arm so as to suspend motion of the part of the surgical robot arm. The restriction of the speed of motion of the surgical robot arm may be a complete restriction of the speed of motion of the terminal arm segment 105t so as to suspend motion of the terminal arm segment.

After the limitation has been placed on the motion of the surgical robot arm, at step 203, the control unit continues to receive an input signal from the surgeon input device 106 but sends an output signal to the surgical robot arm so as to control motion of the surgical robot arm in accordance with the limitation. Therefore, after the clash has been detected and before it is resolved, the motion of the arm requested by the surgeon using the surgeon input device does not translate entirely into the actual motion of the arm.

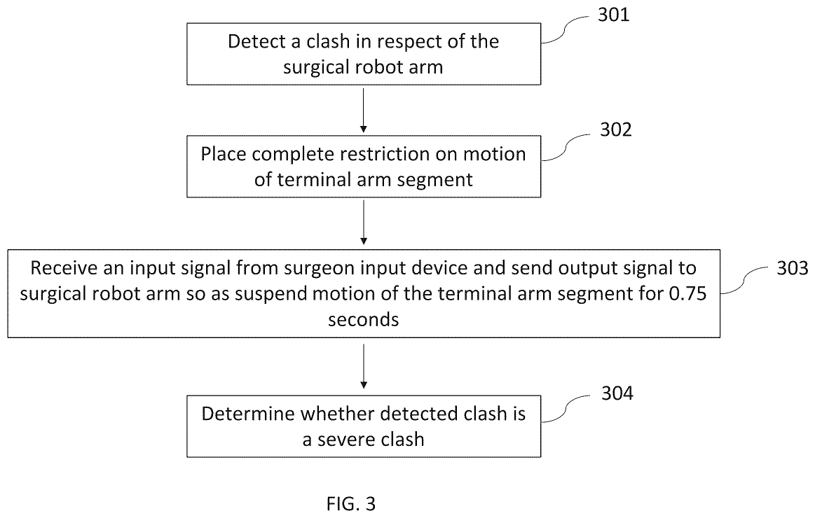

FIGS. 3 to 7 illustrate various stages of an example method which can be performed by the control unit to control the motion of the surgical robot arm after a clash has occurred and before it is resolved.

FIG. 3 illustrates a first stage of the method, the first stage comprising four steps 301 to 304. Broadly, this first stage involves detecting a clash and in response to detecting a clash, holding the arm's terminal segment still for a period of time until the control unit can determine whether or not the clash is a severe clash. In other words, the arm's terminal segment is held still for a period of time as a result of having detected the clash. The first step 301 is to detect a clash in respect of the surgical robot arm. Step 301 may be carried out in the same way as step 201 of FIG. 2, as previously described.

The second step, 302 is to place a complete restriction on motion of the terminal arm segment. Step 303 states that the control unit continues to receive an input signal from the surgeon input device but that the output signal sent from the control unit to the surgical robot arm is so as to suspend motion of the terminal arm segment. Placing a complete restriction on motion of the terminal arm segment and sending output signals to the arm in accordance with that limitation means that motion of the terminal arm segment is suspended. In other words, the position and orientation of the terminal arm segment is fixed.

As described above, under normal operation, the input signal received by the control unit from the surgeon input device constitutes a request for a particular position and orientation of the implement and the control unit is configured to translate the request into an output signal which causes joints in the arm to move to the angles necessary to achieve the requested implement position and orientation. However, once the limitation of the complete restriction of motion of the terminal arm segment has been placed on the motion of the arm, the surgeon may continue to move the hand controllers at the surgeon input device, but such movement will have no effect on the position or orientation of the terminal arm segment (or of the attached implement).

As part of step 303, the motion of the terminal arm segment is suspended for 0.75 seconds. The time period of 0.75 seconds is chosen to account for transient clashes that may trigger a clash momentarily but will resolve themselves soon afterwards without intervention.

Transient clashes may occur in situations in which the arm experiences a short-lived collision with an external object e.g. another arm or a member of the bedside team. Transient clashes may also occur in situations in which the arm is near but slightly under one or more of the thresholds (1) to (7) described above and a small change e.g. in arm position, results in the one or more thresholds being exceeded for a short period of time. According to other examples, the output signals may instruct the surgical robot arm to move in accordance with the limitation for a different length of time. Motion of the terminal arm segment may therefore be suspended for more or less time, for example 0.5 or 1 second. During this time period, the position and orientation of the terminal arm segment does not change.

The robot arm utilised in this method can be moved in a redundant manner meaning that the position of the terminal arm segment (and the attached implement) may be kept constant while the positions of other arm segments are altered. Therefore, other segments and joints of the arm (the movement of which does not affect the terminal arm segment position) may be allowed to move during the time period in which motion of the terminal arm segment is kept constant. For example, the control unit may still be able to receive an input signal as a result of the external force acting on the arm and send an output signal the surgical robot arm to control motion of other segments of the arm. Therefore, due to the redundant nature of the arm, a member of the bedside team may be allowed to reposition another of the arm segments while the position of the terminal arm segment is held constant.

After 0.75 seconds has passed, at step 304, the control unit determines whether the detected clash is a severe clash.

As previously described, detecting the clash may involve determining a value for a variable relating to the motion and or position of the robot arm or implement and comparing that value to a threshold value for the variable. Determining whether the clash is a severe clash may involve comparing the same variable to a higher threshold value for the variable. For example, detecting a clash in respect of the surgical robot arm may comprise one or both of determining a torque value representing the torque at each of the plurality of joints, determining a parameter based on the determined torque value and determining that the parameter exceeds a first torque threshold; and determining a position error of a part of the surgical robot arm or part of the implement and determining that the determined position error exceeds a first position error threshold. Determining whether the detected clash is a severe clash may therefore comprise determining that the determined parameter exceeds a second torque threshold and/or that the determined position error exceeds a third position error threshold, where the second torque threshold is greater than the first torque threshold and the third position error threshold is greater than the first position error threshold. In other words, determining whether the detected clash is a severe clash may involve comparing previously determined variables to new, higher thresholds. As previously described, detecting a clash in respect of the surgical robot arm is used herein to mean detecting that a part of the surgical robot arm or a part of the attached implement meets one or more conditions relating to the motion and/or position of the surgical robot arm or implement.

According to the same example mentioned above, in which the surgical robot arm has 8 joints and the joints are numbered in order starting from the base, the same conditions used in detecting the clash may be used in determining whether the clash is a severe clash.

According to another example, different conditions may be used.

In the example in which the same conditions are used, one or more of those conditions may be compared to different thresholds to determine whether the clash is severe.

Specifically, determining whether the clash is a severe clash may comprise determining that any of the following conditions (1) to (5) is true.
1. That, for any of the first seven joints of the plurality of joints, the magnitude of the gravity compensated adjusted torque about that joint exceeds a threshold value for the joint. The following thresholds may be used.

TABLE 5

| Joint | Threshold (Nm) |
|---|---|
| 1 | 100 |
| 2 | 100 |
| 3 | 60 |
| 4 | 40 |
| 5 | 10 |
| 6 | 10 |
| 7 | 10 |

2. That, for any two joints of the first seven joints in the plurality of joints, the difference between the gravity compensated adjusted torque value at consecutive time intervals exceeds the threshold values for those joints. The following thresholds may be used.

TABLE 6

| Joint | Threshold (Nm) |
|---|---|
| 1 | 0.2 |
| 2 | 0.3 |
| 3 | 0.3 |
| 4 | 0.3 |
| 5 | 0.1 |
| 6 | 0.03 |
| 7 | 0.03 |

3. That, the difference between the joint angle derived from the input actuator position and the actual output joint angle, for one or both of joints 6 and 7, exceeds a threshold value for the joint. The following thresholds may be used.

TABLE 7

| Joint | Threshold (rad) |
|---|---|
| 6 | 0.08 |
| 7 | 0.08 |

4. That, for one or more joints of joints 3 to 8, the difference between the requested joint angle and the actual output joint angle exceeds a threshold value for the joint. The following thresholds may be used.

TABLE 8

| Joint | Threshold (rad) |
|---|---|
| 3 | 0.02 |
| 4 | 0.01 |
| 5 | 0.03 |
| 6 | 0.04 |
| 7 | 0.04 |
| 8 | 0.2 |

5. That, the distance between the requested position of the tip of the implement and the actual position of the tip of the implement exceeds 20 mm.

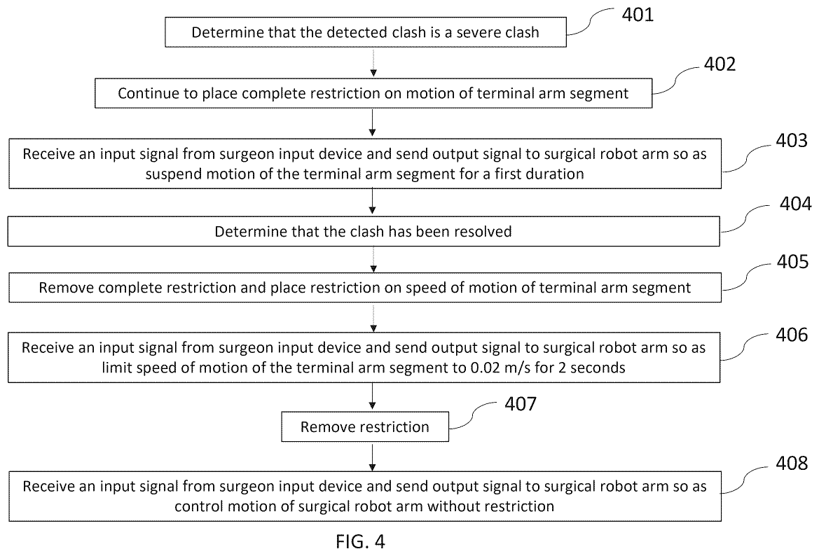

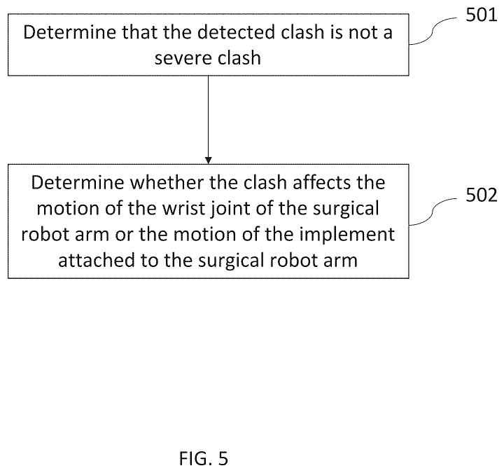

If it is determined that the detected clash is a severe clash, the control unit proceeds to perform the method illustrated in FIG. 4. If it is determined that the detected clash is not a severe clash, the control unit proceeds to perform the method illustrated in FIG. 5.

FIG. 4 illustrates an example of a method that may be performed by the control unit once it has performed step 304 (of FIG. 3) and determined (at step 401) that the detected clash is a severe clash according to the method described above. Once the control unit has determined that the detected clash is a severe clash, the unit continues to place the limitation of complete restriction on motion of the terminal arm segment (at step 402) and continues to receive an input signal from the surgeon input device and send an output signal to the surgical robot arm so as to suspend motion of the terminal arm segment (at step 403). In other words, the control unit continues to instruct the surgical robot arm to keep the position and orientation of the terminal arm segment fixed. The control unit sends output signals in accordance with this limitation for a first duration. Motion of the terminal arm segment is thus suspended for a first duration.

When the clash is determined to be severe, this means that it is important that the terminal arm segment (and the implement) are held in a fixed position to avoid inflicting damage on the patient. In this scenario it is therefore not advisable for the surgeon to resolve the clash by moving the terminal arm segment of the arm. Thus, when the clash is determined to be severe, the clash may only be resolved by a member of the bedside team imparting an external force on the arm or actuating a control (e.g. a button) on the arm.

As previously explained, the arm utilised in this method can move in a redundant manner meaning that the positions of other arm segments can be altered while the position of the terminal arm segment (and the attached implement) are kept constant. After the clash is determined to be severe at step 401, during the first duration, the control unit is still able to receive an input signal as a result of an external force acting on the arm and send an output signal the surgical robot arm to control motion of segments of the arm that are not the terminal arm segment. Therefore, due to the redundant nature of the arm, a member of the bedside team can reposition one or more arm segments (that are not the terminal arm segment) while the position of the terminal arm segment is held constant so as to resolve the clash. The motion of the terminal segment may be suspended until the clash has been resolved by a member of the bedside team. In other words, the first duration may be the length of time between determining that the clash is a severe clash and determining that the clash has been resolved. Determining that the clash has been resolved may comprise determining that a clash that was previously detected is no longer present.

As mentioned above, the control unit may continue to check for clashes at regular time intervals. For example, the control unit may continue to check for clashes throughout steps 402 and 403. This enables the control unit to determine when a detected clash has been resolved. At step 403, the control unit determines that the clash has been resolved.

Determining that a clash has been resolved may require detecting that no parts of the surgical robot arm or parts of the attached implement meet the one or more conditions relating to the motion and/or position of the surgical robot arm or implement described above. For example, determining that a clash has been resolved may comprise determining a value for a variable relating to the motion and or position of the robot arm or implement, comparing that value to a threshold value for the variable and determining that the value for the variable does not exceed the threshold value for the variable. Determining that a clash has been resolved may comprise repeating this process for each variable relating to the motion and or position of the robot arm or implement and determining that none of the values of these variables exceed their respective thresholds.

Determining that a clash in respect of the surgical robot arm has been resolved may comprise determining a torque value representing the torque at each of the plurality of joints, determining a parameter based on the determined torque value, determining that the parameter does not exceed any thresholds associated with any of the joints, determining a position error of a part of the surgical robot arm or part of the implement and determining that the determined position error does not exceed any thresholds associated with any of the parts of the arm or implement. According to one example, determining that a clash has been resolved may comprise determining that none of conditions (1) to (7) are true. Alternatively determining that a clash has been resolved may comprise determining that the one or more conditions of conditions (1) to (7) previously determined to be true are no longer true.

Step 405 is therefore performed after the first duration. The control unit performs step 405 after it has determined that the clash has been resolved. At step 405, the control unit removes the complete restriction of motion of the terminal arm segment. At step 405, the control unit places a (different) limitation on the motion of the surgical robot arm. Specifically, the control unit places a restriction on the speed of motion of the terminal arm segment. In the example seen in FIG. 4, the limitation is a restriction of the speed of the terminal arm segment to a maximum of 0.02 m/s. According to other examples, the speed of the terminal arm segment may be limited to 0.01 m/s, 0.015 m/s, 0.025 m/s, 0.03 m/s.

At step 406, the control unit continues to receive an input signal from the surgeon input device. The control unit additionally sends an output signal to the surgical robot arm so as to limit the speed of motion of the terminal arm segment in accordance with the limitation. In other words, the control unit instructs the surgical robot arm to move such that the terminal arm segment can move at a maximum speed of 0.02 m/s. While the speed of motion of the terminal arm segment is limited, no limitations are placed on the direction of motion of the terminal arm segment during this period. The surgeon is therefore able to use the surgeon input device to position the terminal arm segment at any desired position and orientation but any movement of the terminal arm segment is limited to a maximum speed of 0.02 m/s.

This step of the process is advantageous as it ensures that when the surgeon is again able to cause motion of the surgical robot arm, that they don't straightaway cause any damage to the patient as a result of the moving the hand controllers too quickly.

In the example shown in FIG. 4, the control unit continues to send an output signal to the surgical robot arm to limit the speed of motion of the terminal arm segment for 2 seconds.

The control unit may limit the speed of the terminal arm segment to 0.02 m/s until 2 seconds has passed since it was determined that the clash has been resolved. In other examples, the speed of motion of the terminal arm segment may be limited for a longer or shorter period of time. For example, the control unit may send an output signal to the surgical robot arm so as to limit the speed of motion of the terminal arm segment for 1 second or 3 seconds after it is determined that the clash has been resolved.

After 2 seconds have passed since the clash was resolved, at step 407, the control unit removes the restriction. The restriction may be removed instantaneously. Alternatively, the restriction may be removed gradually over a period of time such that the permitted speed of motion of the terminal arm segment is slowly ramped up from 0.02 m/s to the normal permitted operating speed. At step 408, the control unit continues to receive an input signal from surgeon input device but now sends an output signal to the surgical robot arm so as control motion of surgical robot arm without restriction. After step 407, the control unit does not place any limitations on the motion of the surgical robot arm. Therefore, at step 408, the control unit returns to normal operation. In other words, the control unit operates as it did before the clash was detected. Were a further clash to be detected after step 408, the control unit would restart the method beginning at step 301. It should be noted that any changes to the permitted speed of motion of the arm described herein may be performed instantaneously or may be implemented gradually over a period of time.

FIG. 5 illustrates an example of steps of the method that may be performed by the control unit once it has performed step 304 (of FIG. 3) and determined (at step 501) that the detected clash is not a severe clash. After it has been determined that the detected clash is not a severe clash, the control unit performs step 502. At step 502, the control unit determines whether the clash affects the motion of the wrist joint of the surgical robot arm or the motion of the implement attached to the surgical robot arm. The following description relates to an example in which the method involves determining whether the clash affects the motion of the wrist joint of the surgical robot arm, but it will be understood that according to other examples, the method may involve determining whether the clash affects the motion of any particular joint of the plurality of joints in the surgical robot arm.

Determining whether the clash affects the motion of the wrist joint of the surgical robot arm or the motion of the implement attached to the surgical robot arm may comprise determining the force at the wrist joint and determining that the determined wrist force exceeds a wrist force threshold. Determining the force at the wrist joint may comprise receiving measurements from one or more of the torque sensors on the arm. The wrist force threshold may be between 20N and 50N. The wrist force threshold may be a force of 35N.

Determining whether the clash affects the motion of the wrist or implement may comprise determining a position error of the distal end of the implement and determining that the determined position error exceeds an implement position error threshold. The position error may be the distance between the requested position of the tip of the implement (as a result of the signal received from the surgeon input device) and the actual position of the tip of the implement, which may be determined by one or more of the position sensors present on the arm. The implement position error threshold may between 10 mm and 20 mm. The implement position error threshold may be a distance of 15 mm. Determining that the clash affects the motion of the wrist joint of the surgical robot arm or the motion of the implement attached to the arm may comprise determining that the force at the wrist joint of the arm exceeds the wrist force threshold and/or that the position error of the distal end of the implement exceeds the implement position error threshold.

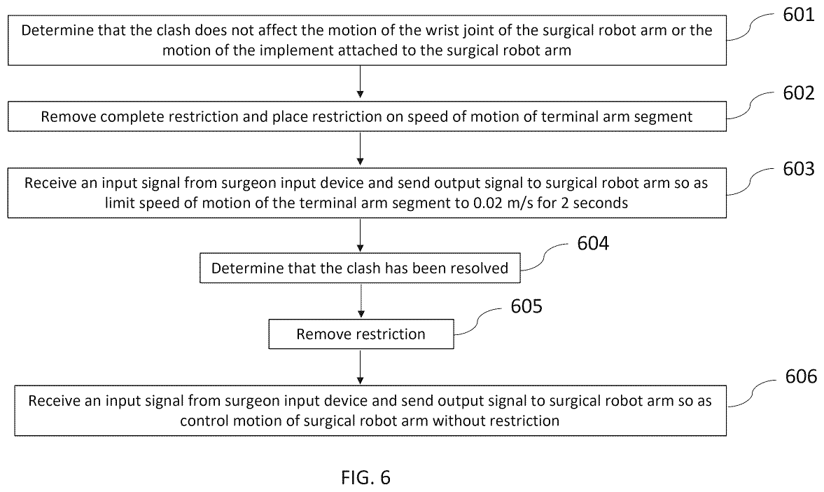

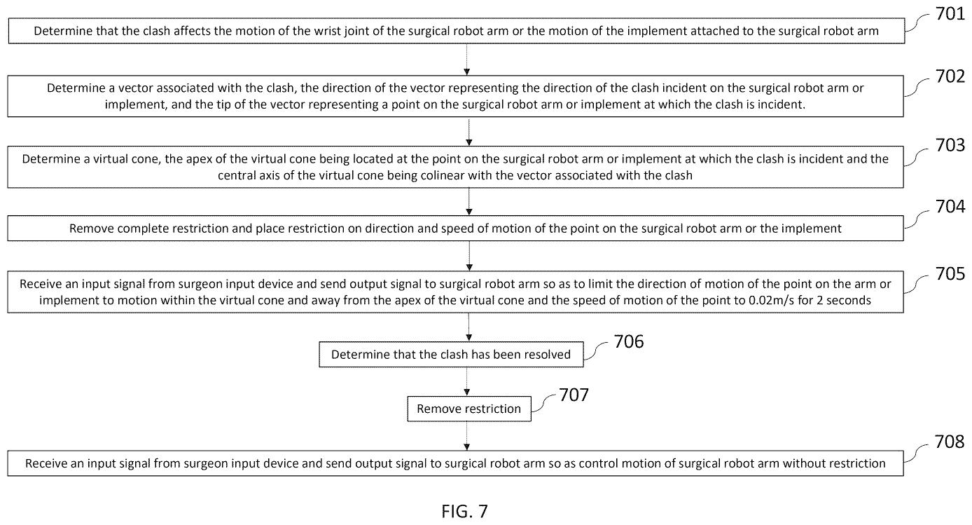

If at step 502, it is determined that the clash does not affect the motion of the wrist joint of the surgical robot arm or the motion of the implement attached to the surgical robot arm, the control unit proceeds to perform the method illustrated in FIG. 6. If it is determined that the clash does affect the motion of the wrist joint of the surgical robot arm or the motion of the implement attached to the surgical robot arm, the control unit proceeds to perform the method illustrated in FIG. 7.

FIG. 6 illustrates an example of a method that may be performed by the control unit when it has determined (at step 601) that the clash does not affect the motion of the wrist joint of the surgical robot arm or the motion of the implement attached to the surgical robot arm.

An example of such a clash may be a collision between the elbow joint 104e and a part of another surgical robot arm or other piece of apparatus. After the control unit has determined that the clash does not affect the motion of the wrist joint of the surgical robot arm or the motion of the implement attached to the surgical robot arm, at step 602, the control unit removes the complete restriction on the speed of motion of the terminal arm segment. At step 602, the control unit places a (different) limitation on the motion of the surgical robot arm. Specifically, the control unit places a restriction on the speed of motion of the terminal arm segment. In the example seen in FIG. 6, the limitation is a restriction of the speed of the terminal arm segment to a maximum of 0.02 m/s. According to other examples, the speed of the terminal arm segment may be limited to 0.01 m/s, 0.015 m/s, 0.025 m/s, 0.03 m/s.

At step 603, the control unit continues to receive an input signal from the surgeon input device. The control unit additionally sends an output signal to the surgical robot arm so as to cause the speed of motion of the terminal arm segment to be limited in accordance with the limitation. In other words, the control unit instructs the surgical robot arm to move such that the terminal arm segment can move at a maximum speed of 0.02 m/s. While the speed of motion of the terminal arm segment is limited, no restrictions are placed on the direction of motion of the terminal arm segment during this period. The surgeon is therefore able to use the surgeon input device to position the terminal arm segment at any desired position and orientation but any movement of the terminal arm segment is limited to a speed of 0.02 m/s.

After it has been determined that the clash is not a severe clash and that the clash does affect the motion of the wrist joint of the surgical robot arm or the motion of the implement attached to the surgical robot arm, the control unit is able to receive an input signal from the surgeon input device and send an output signal to the surgical robot arm so as to limit the speed of motion of the terminal arm segment to a maximum of 0.02 m/s. The control unit is also able to receive an input signal as a result of an external force acting on a part of the arm and send an output signal to the surgical robot arm to control motion of the arm.

Motion of the terminal arm segment is limited to 0.02 m/s, however other arm segments may be controlled to move at speeds greater than 0.02 m/s.

As previously explained, when the clash is determined to be severe in FIG. 4, the control unit controls the surgical robot arm such that the clash may only be resolved by a member of the bedside team imparting a force on the arm or by actuating a control on the arm. In contrast, when the clash is determined not to be severe, the clash may be resolved by a member of the bedside team imparting an external force on the arm or actuating a control on the arm or by the surgeon using the surgeon input device to move the terminal arm segment. The surgeon is able to move the terminal arm segment in any direction at a speed of up to 0.02 m/s to try to resolve the clash.

At step 604, the control unit determines that the clash has been resolved. The control unit may determine that the clash has been resolved in accordance with any of the previously described methods.

Step 605 is performed when 2 seconds have passed since determining that the clash has been resolved. At step 605, the restriction on the motion of the surgical robot arm is removed. According to other examples, the control unit may remove the restriction more quickly or more slowly after detecting that the clash has been resolved. For example, the control unit may send an output signal to the surgical robot arm so as to limit the speed of motion of the terminal arm segment for 1 second or 3 seconds after it is determined that the clash has been resolved. Step 605 of removing the restriction may be performed instantaneously. Alternatively, the restriction may be removed gradually over a period of time such that that the permitted speed of motion of the terminal arm segment is slowly ramped up from 0.02 m/s to the normal permitted operating speed.

At step 606, the control unit continues to receive an input signal from the surgeon input device but now sends an output signal to the surgical robot arm so as control motion of surgical robot arm without restriction. After step 605, the control unit does not place any limitations on the motion of the surgical robot arm. Therefore, at step 606, the control unit returns to normal operation. In other words, the control unit operates as it did before the clash was detected. Were a further clash to be detected after step 606, the control unit would restart the method beginning at step 301.

FIG. 7 illustrates an example of a method that may be performed by the control unit when it has determined (at step 701) that the clash does affect the motion of the wrist joint of the surgical robot arm or the motion of the implement attached to the surgical robot arm. An example of a scenario in which the clash affects the motion of the wrist joint or implement is a collision between the terminal arm segment of the surgical robot arm and another robot arm or other piece of apparatus.

After the control unit has determined that the clash does affect the motion of the wrist joint of the surgical robot arm or the motion of the implement attached to the surgical robot arm, at step 702, the control unit determines a vector associated with the clash. The vector associated with the clash is indicative of the direction of the clash. The vector associated with the clash is indicative of the point on the arm or implement at which the clash is incident.

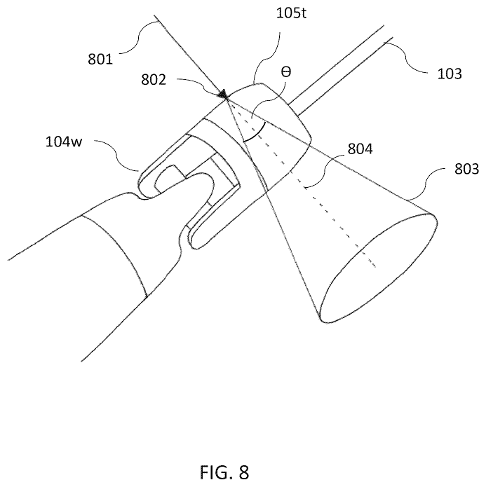

FIG. 8 illustrates the distal end of the surgical robot arm seen in FIG. 1 including the terminal arm segment 105t and the wrist joint 104w. FIG. 8 also illustrates an implement 103 attached to the terminal arm segment 105t. FIG. 8 shows a vector 801 which is associated with a clash incident on the terminal arm segment 105t. The vector associated with the clash has a direction which represents the direction of the clash incident on the surgical robot arm or implement. The direction of the vector 801 represents the direction of the clash incident on the terminal arm segment 105t. The vector associated with the clash has a tip which represents a point on the surgical robot arm or implement at which the clash is incident. The tip 802 of vector 801 represents a point on the terminal arm segment 105t at which the clash is incident. The vector is associated with a clash which has been determined as affecting the motion of the wrist joint of the surgical robot arm or the motion of the implement attached to the surgical robot arm. The point on the surgical robot arm or implement at which the clash is incident may therefore be any point at which a clash incident on that point would affect the motion of the wrist or implement. The point at which the clash is incident may therefore be a point on the wrist joint, the terminal arm segment, an arm segment connected to the wrist joint, or the implement.

The control unit may determine a vector associated with the clash according to the following method.

As previously mentioned, the surgical robot arm 102 may comprise a series of torque sensors positioned at each joint 104 of the arm, each torque sensor being configured to measure the torque exerted on the joint at which it is located. The surgical robot arm 102 is configured to send the measurements of the torques exerted at the joints to the control unit 107. The control unit uses the torque measurements received from the torque sensors to determine a vector associated with the clash.

Determining a vector associated with the clash may comprise receiving a measurement of the torque at each of the plurality of joints from the surgical robot arm. The control unit may process the received torque measurements so as to account for torques which do not result from the clash. For example, the received torque measurements may be adjusted so as to remove torques resulting from gravity due to the mass of the robot arm, torques resulting from internal gear train stretching and/or reaction torques resulting from the attached implement interacting with the port and surgical site. The result of said processing is only the torque which results from the clash incident on the arm or implement. In other words, the control unit determines a torque vector τ comprising the torque at each of the plurality of joints which results from the clash.

The control unit may then calculate a resultant force ƒ which acts on the point of the arm or implement at which the clash is incident as a result of the clash occurring. Calculating the resultant force ƒ may comprise calculating a force vector ƒ corresponding to the resultant force acting at a series of points along the robot arm using the torques τ. The force vector ƒ may be calculated by applying the principle of virtual work to the determined joint torques τ.

The principle of virtual work states that moving a point P on the arm or implement a distance ∂p\hat{x} (where \hat{x} is a unit vector) requires a joint J to move by an angle ∂θ_{J}, so if a force is applied at point P in the direction of \hat{x}, the torque τ_{J} seen by the joint J is proportional to the displacement over θ_{J}. This can be written in a Jacobian matrix Jₚ.

$$τ = ( \begin{matrix}{τ}_{1} \\ ⋮ \\ {τ}_{n}\end{matrix} ) = ( \begin{matrix}\frac{\partial {p}_{x}}{\partial {θ}_{1}} & … & \frac{\partial {p}_{z}}{\partial {θ}_{1}} \\ ⋮ & ⋱ & ⋮ \\ \frac{\partial {p}_{x}}{\partial {θ}_{n}} & … & \frac{\partial {p}_{z}}{\partial {θ}_{n}}\end{matrix} ) f = {J}_{p} f \qquad ( equation 1 )$$

where τ is a vector of the torques acting at each of the n joints of the surgical robot arm, τ is the torque acting at each joint, ∂pₓ is the distance moved by a series of points along the robot arm or implement in the direction x, ∂θₙ is the angle moved by the joint n and ƒ is a force vector corresponding to the resultant force acting at the series of points along the robot arm or implement. The Jacobian matrix Jₚ may be a function of the geometry and pose of the arm.

The result of resolving in directions x, y and z is:

$$τ = ( \begin{matrix}{τ}_{1} \\ ⋮ \\ {τ}_{n}\end{matrix} ) = ( \begin{matrix}\frac{\partial {p}_{x}}{\partial {θ}_{1}} & … & \frac{\partial {p}_{z}}{\partial {θ}_{1}} \\ ⋮ & ⋱ & ⋮ \\ \frac{\partial {p}_{x}}{\partial {θ}_{n}} & … & \frac{\partial {p}_{z}}{\partial {θ}_{n}}\end{matrix} ) ( \begin{matrix}{\hat{d}}_{x} \\ {\hat{d}}_{y} \\ {\hat{d}}_{z}\end{matrix} ) f = {J}_{p} \hat{d} f = ( \begin{matrix}\frac{\partial {p}_{\hat{d}}}{\partial {θ}_{1}} \\ ⋮ \\ \frac{\partial {p}_{\hat{d}}}{\partial {θ}_{n}}\end{matrix} ) f = {J}_{p , \hat{d}} f \qquad ( equation 2 )$$

which can be rearranged to form:

$$f = K τ \qquad ( equation 3 )$$

where K is the inverse of J_{p,\hat{d}}.

Determining a vector associated with the clash may therefore comprise calculating the Jacobian matrix of displacement and joint angular displacement J_{p,\hat{d}}, finding the inverse of J_{p,d̅} to obtain the Jacobian K and multiplying K by the torques acting at each of the n joints of the surgical robot arm to calculate a vector ƒ representing the resultant forces which act at a series of points along the robot arm.

As previously mentioned, the surgical robot arm 102 illustrated in FIG. 1 may comprise a position sensor (not shown) located at a plurality of points along the surgical robot arm. Each position sensor is configured to measure the position of the point on the surgical robot arm at which it is located. The surgical robot arm may be configured to send the measurements of position at the points along the arm to the control unit. The control unit may be configured to infer angles of each of the joints n from the measurements of position by the position sensors and knowledge of the geometry of the surgical robot arm. The Jacobian Jₚ may therefore be generated by the control unit according to measurements received from the surgical robot arm.

As explained above, the result of multiplying the vector of the torques acting at each of the n joints of the surgical robot arm τ by the inverse Jacobian K is a vector ƒ corresponding to the resultant force acting at the series of points along the robot arm. The control unit may be configured to deduce from the vector ƒ the point on the arm or implement at which the resultant force acts. The control unit may therefore be configured to deduce from the vector ƒ the point on the arm or implement at which the clash was incident. The calculated resultant force is therefore representative of the clash. The control unit is configured to use the vector ƒ to determine the vector associated with clash. The vector associated with the clash may be determined to have a direction which is the same as the direction of the calculated resultant force. The tip of the vector associated with the clash may be determined to have a position which is the same as the point on the arm or implement at which the resultant force has been found to act.

Once the vector associated with the clash has been determined, at step 703, the control unit determines a virtual cone. FIG. 8 illustrates a virtual cone 803. The virtual cone has an apex which is located at the point on the surgical robot arm or implement at which the clash is incident. In other words, the apex of the virtual cone is located at the tip of the vector associated with the clash (i.e. at point 802 in FIG. 8). The virtual cone has a central axis 804. The central axis of the virtual cone is colinear with the vector associated with the clash. As seen in FIG. 8, central axis 804 of virtual cone 803 is colinear with the vector 801.

At step 704, the control unit removes the complete restriction on the speed of motion of the terminal arm segment. At step 704, the control unit places a (different) limitation on the motion of the surgical robot arm. The control unit places a restriction on the direction and speed of motion of the point on the surgical robot arm or implement at which the clash is incident. The limitation is a restriction of the direction of motion in which the point on the arm or implement at which the clash is incident (801 in FIG. 8) can be controlled by the surgeon to move. Specifically, the limitation is a restriction of the direction of motion of said point to motion within the virtual cone in a direction that is away from the apex of the virtual cone. The limitation is also a restriction of the speed of motion of said point. In the example of FIG. 7, the limitation is a restriction of the speed of the terminal arm segment to a maximum of 0.02 m/s.

At step 705, the control unit continues to receive an input signal from the surgeon input device and sends an output signal to the surgical robot arm so as to limit the direction of motion of the point on the arm or implement to motion within the virtual cone and away from the apex of the virtual cone and the speed of motion of the point to a maximum of 0.02 m/s. The purpose of allowing movement only within the virtual cone is that the surgeon is able to control the point to move only in a way that will help to resolve the clash but cannot cause the point to move in such a way that will not resolve the clash (or reinforce the clash).

Thus, in the example seen in FIG. 8, the surgeon is able to use the surgeon input device to move the terminal arm segment in a direction which will help to resolve the clash but any movement of the terminal arm segment is limited to a maximum speed of 0.02 m/s. In the example seen in FIG. 8, the surgeon is able to move the terminal arm segment such that the point 802 on the terminal arm segment moves away from the starting position of point 802 within the virtual cone 803 at a speed of 0.02 m/s to try to resolve the clash.

As previously explained, the surgeon input device 106 may comprise hand controllers which the surgeon can move to cause motion of the surgical robot arm. According to the method of FIG. 7, after step 704 has been performed and the control unit has placed a restriction on the direction and speed of motion of the surgical robot arm, the surgeon retains the ability to move the hand controllers freely, however only motion of the hand controllers which translates into motion of the point 802 within the virtual cone 803 and in a direction away from the cone's apex 802 will cause motion of the point. Other motion of the hand controllers by the surgeon will not translate to any movement of point 802. Only motion of the hand controllers which causes motion of the point that would help to the resolve the clash will result in any movement of the surgical robot arm. In other words, at step 705, the control unit may receive an input signal from the surgeon input device representing desired motion both inside and outside the virtual cone but will only send an output signal to the surgical robot arm which causes motion of the point within the virtual cone and away from the apex of the virtual cone.

The apex angle is illustrated as angle θ in FIG. 8. The apex angle of the virtual cone is the angle at the cone's apex between opposite sides of the cone. In order for motion of the point 802 within the cone and away from the cone's apex to result in the clash being resolved, the apex angle must be equal to or less than 180 degrees. If the apex angle is very small (for example less than 40 degrees), i.e. if the cone is very narrow, the permitted motion of the point 802 is very limited meaning that it becomes more difficult for the surgeon to cause any motion of the surgical robot arm. It is therefore more difficult for the surgeon to resolve the clash and places more reliance on the bedside team to resolve the clash. This can cause the surgeon to become frustrated and reduce overall surgeon satisfaction with the system.

However, when the apex angle is equal to or close to 180 degrees i.e. when the cone is very wide, this can result in other problems. For example, when the clash results from a collision between the point 802 and another surface, enabling the surgeon to move the point within a cone having an apex angle of 180 may cause the point to slide along the surface until it reaches the end of the surface e.g. an edge. At this point it has been found that the point may “fall off” the surface leading to a jolt in the movement of the distal end of the arm. Such a jolt can cause damage to the patient due to the sudden uncontrolled movement of the implement. It is therefore desirable that the apex angle of the cone is not close to 180 degrees. It has been found to be optimal that the apex angle of the virtual cone is within a range from 40 degrees to 100 degrees. In particular, the apex angle of the virtual cone may be 60 degrees. Other suitable apex angles may be 100, 90, 80, 70, 50 or 40 degrees.

As previously explained, when the clash is determined to be severe, the control unit controls the surgical robot arm such that the clash may only be resolved by a member of the bedside team imparting a force on the arm or actuating a control on the arm. In contrast, when the clash is determined not to be severe, the clash may be resolved by a member of the bedside team imparting an external force on the arm or by actuating a control on the arm or by the surgeon using the surgeon input device to move the terminal arm segment. The surgeon is able to move the point on the arm or implement at which the clash is incident within the virtual cone in a direction away from the apex of the cone in any direction at a speed of 0.02 m/s to try to resolve the clash.

At step 706, the control unit determines that the clash has been resolved. The control unit may determine that the clash has been resolved according to previously described methods.

Step 707 is performed when 2 seconds have passed since determining that the clash has been resolved. At step 707, the restriction on the motion of the surgical robot arm is removed. According to other examples, the control unit may remove the restriction more quickly or more slowly after detecting that the clash has been resolved. For example, the control unit may send an output signal to the surgical robot arm so as to limit the speed of motion of the terminal arm segment for 1 second or 3 seconds after it is determined that the clash has been resolved. Step 707 of removing the restriction may be performed instantaneously. Alternatively, the restriction may be removed gradually over a period of time such that that the permitted speed of motion of the terminal arm segment is slowly ramped up from 0.02 m/s to the normal permitted operating speed.

At step 708, the control unit continues to receive an input signal from surgeon input device but now sends an output signal to the surgical robot arm so as control motion of surgical robot arm without restriction. After step 707, the control unit does not place any limitations on the motion of the surgical robot arm. Therefore, at step 708, the control unit returns to normal operation. In other words, the control unit operates as it did before the clash was detected. Were a further clash to be detected after step 708, the control unit would restart the method beginning at step 301.

As previously mentioned, the system 100 shown in FIG. 1 may be part of a larger system including one or more further surgical robot arms. In such a system, the control unit 107 is configured to receive an input signal from the surgeon input device with respect of each of the surgical robot arms. The software stored in memory 109 of the control unit is configured to respond to those inputs and the processor 108 is configured to execute the software to send an output signal to the actuators of any of the one or more further surgical robot arms to cause the joints of the respective arm to move in accordance with the input signal from the surgeon input device. The surgeon is thereby able to control the motion of each of the surgical robot arms using the surgeon input device. As well as the previously described control unit 107 and surgeon command interface 106, a larger surgical system may include any number of surgical robot arms connected to a surgical instrument (instrument arms) and any number of surgical robot arms connected to an endoscope (endoscope arms). The endoscope provides a signal to the display of the surgeon command interface 106 so that the surgeon is able to view the surgical site.

The control unit is configured to receive an input signal from the surgeon input device for controlling any of the surgical robot arms in the system. The control unit is configured to send an output signal to any of the robot arms to cause motion of the respective arm. When a clash is detected with respect to an endoscope arm, in response to detecting the clash (i.e. as a result of having detected the clash), the control unit is configured to place a limitation on the motion of the endoscope arm and some or all of the other surgical robot arms in the system. The limitation may be a complete restriction of the speed of motion of the terminal arm segment of some or all of the surgical robot arms in the system so that movement of the said terminal arm segments is suspended.

The control unit is able to distinguish between an instrument arm and an endoscope arm using various means. The control unit is therefore able to distinguish between a clash with respect of an instrument arm and a clash in respect of an endoscope arm.

If a clash occurs with respect to an endoscope arm, the signal received at the control unit from the endoscope may be impeded such that the surgeon's ability to view the surgical site at the surgeon command interface is negatively affected. In order to ensure that no damage is caused to a patient as a result and improve the safety of the surgical procedure, when a clash is incident on an endoscope arm, it is desirable to prevent motion of the endoscope and any surgical instruments in the system. It is therefore advantageous to limit the motion of the terminal arm segment of every robot arm in the system.

According to one example in which the system includes one endoscope arm and three instrument arms and the control unit detects a clash in respect of the endoscope arm, the control unit proceeds to follow the methods illustrated in FIGS. 3 to 7 and described above, however any step involving placing or removing a complete restriction of the robot arm's terminal arm segment is replaced with the same step being applied to all four robot arms in the system. For example, at step 302, in addition to placing a complete restriction on the motion of the terminal arm segment of the endoscope arm, the control unit places the same restriction on motion of the terminal arm segment of the three instrument arms. Step 303 is therefore performed by the control unit so as to suspend motion of the terminal arm segment of all four arms for 0.75 seconds before determining whether the clash is a severe clash at step 304. In other examples, the control may only cause motion of the terminal arm segment of some of the robot arms in the system to be suspended.

The memory 109 in FIG. 1 may be a non-transitory computer readable storage medium which stores computer readable instructions that, when executed at a computer system, for example processor 108, cause the computer system to perform any of the methods described herein.

It is to be understood that the robot arm described herein could be for purposes other than surgery. For example, the surgical robot arm could be controlled for manipulating tissue, which is not part of a patient, e.g. for manipulating tissue of a cadaver or of any other object. As another example, the robot arm could control a viewing instrument for viewing inside a manufactured article such as a car engine.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A control unit for controlling motion of a surgical robot arm, the control unit being configured to receive an input signal from a surgeon input device for controlling the surgical robot arm and send an output signal to the surgical robot arm to cause motion of the surgical robot arm, the control unit being further configured to:
detect a clash in respect of the surgical robot arm;
in response to detecting the clash, place a limitation on the motion of the surgical robot arm; and
receive an input signal from the surgeon input device for controlling the surgical robot arm and send an output signal to the surgical robot arm so as to control motion of the surgical robot arm in accordance with the limitation.

2. The control unit of claim 1, wherein:
the surgical robot arm comprises a plurality of arm segments connected by a plurality of driven joints extending between a base and a terminal arm segment; and
the terminal segment of the surgical robot arm is configured to connect to an implement.

3. The control unit of claim 1, wherein the surgical robot arm comprises a plurality of arm segments connected by a plurality of driven joints extending between a base and a terminal arm segment and the limitation comprises a restriction of the speed of motion of the terminal arm segment to a maximum value, the maximum value being greater than zero.

4. The control unit of claim 2 wherein the clash that is detected in respect of the surgical robot arm is a clash on the surgical robot arm or a clash on an implement connected to the surgical robot arm.

5. (canceled)

6. The control unit of claim 2, wherein detecting a clash in respect of the surgical robot arm comprises one or both of:
determining a torque value representing the torque at each of the plurality of joints, determining a parameter based on the determined torque value and determining that the parameter exceeds a first torque threshold; and
determining a position error of a part of the surgical robot arm or part of the implement connected to the surgical robot arm, and determining that the determined position error exceeds a first position error threshold.

7. The control unit of claim 6, wherein the control unit is further configured to determine whether the detected clash in respect of the surgical robot arm is a severe clash, wherein determining whether the detected clash is a severe clash comprises:
determining that the determined parameter exceeds a second torque threshold and/or that the determined position error exceeds a second position error threshold,
wherein the second torque threshold is greater than the first torque threshold and the second position error threshold is greater than the first position error threshold.

8. The control unit of claim 7, wherein the control unit is configured to, in response to determining that the detected clash is a severe clash, place a first limitation on the motion of the surgical robot arm for a first duration, wherein the first limitation is a complete restriction of the speed of motion of terminal arm segment,
wherein the control unit is further configured to place a second limitation on the motion of the surgical robot arm for a second duration after the first duration, wherein the second limitation is a restriction of the speed of motion of the terminal arm segment to a maximum value, the maximum value being greater than zero.

9. (canceled)

10. The control unit of claim 1, wherein the limitation comprises one or both of a restriction of the speed of motion of the surgical robot arm and a restriction of the direction of motion of the surgical robot arm.

11. The control unit of claim 10, wherein the surgical robot arm comprises:
a plurality of arm segments connected by a plurality of driven joints extending between a base and a terminal arm segment; and
the terminal segment of the surgical robot arm is configured to connect to an implement; and
the restriction of the speed of motion of the surgical robot arm comprises a complete restriction of the speed of motion of the terminal arm segment so as to suspend motion of the terminal arm segment.

12. (canceled)

13. The control unit of claim 10, wherein the surgical robot arm comprises:
a plurality of arm segments connected by a plurality of driven joints extending between a base and a terminal arm segment; and
the terminal segment of the surgical robot arm is configured to connect to an implement; and
the restriction of the speed of motion of the surgical robot arm is a restriction of the speed of motion of the terminal arm segment to a maximum value, the maximum value being greater than zero.

14. The control unit of claim 2, the control unit being further configured to determine whether the clash affects the motion of a particular joint of the surgical robot arm or the motion of the implement, wherein determining whether the clash affects the motion of the particular joint of the surgical robot arm or the motion of the implement comprises one or both of:
determining a force at the particular joint, and determining that the determined force exceeds a force threshold; and
determining a position error of the distal end of the implement, and determining that the determined position error exceeds an implement position error threshold.

15. The control unit of claim 14, wherein the control unit is configured to, in response to determining that the clash affects the motion of the particular joint of the surgical robot arm or the motion of the implement, determine a vector associated with the clash.

16. The control unit of claim 15, wherein the vector associated with the clash has a direction and a tip, the direction of the vector represents the direction of the clash incident on the surgical robot arm or implement, and the tip of the vector represents a point on the surgical robot arm or implement at which the clash is incident.

17. The control unit of claim 16, wherein the point on the surgical robot arm or implement at which the clash is incident is a point on the particular joint, the terminal arm segment, an arm segment connected to the particular joint, or the implement.

18. The control unit of claim 16, wherein the limitation is a restriction of the direction of motion of the point on the surgical robot arm or implement to motion in a direction within a virtual cone and away from an apex of the virtual cone, the apex of the virtual cone being located at the point on the surgical robot arm or implement at which the clash is incident and the central axis of the virtual cone being colinear with the vector associated with the clash.

19. (canceled)

20. The control unit of claim 14, wherein:
the plurality of driven joints comprise a wrist joint which is the most distal of the driven joints that is not a roll joint; and
the particular joint of the plurality of driven joints is the wrist joint.

21. (canceled)

22. A surgical system comprising a surgical robot arm and a control unit for controlling motion of the surgical robot arm, the control unit being configured to receive an input signal from a surgeon input device for controlling the surgical robot arm and send an output signal to the surgical robot arm to cause motion of the surgical robot arm, the control unit being further configured to:
detect a clash in respect of the surgical robot arm;
in response to detecting the clash, place a limitation on the motion of the surgical robot arm; and
receive an input signal from the surgeon input device for controlling the surgical robot arm and send an output signal to the surgical robot arm so as to control motion of the surgical robot arm in accordance with the limitation.

23. (canceled)

24. The surgical system of claim 22, wherein the surgical robot arm comprises a terminal arm segment connected to an endoscope and the system comprises one or more further surgical robot arms and the control unit is configured to:
for each of the one or more further surgical robot arms, receive an input signal from the surgeon input device for controlling the surgical robot arm and send an output signal to the surgical robot arm to cause motion of the surgical robot arm; and
in response to detecting the clash, place a limitation on the motion of each of the one or more further surgical robot arms, wherein the limitation comprises a complete restriction of the speed of motion of the terminal arm segment of that one of the one or more further surgical robot arms so as to suspend motion of the terminal arm segment of that one of the one or more further surgical robot arms.

25. A method for controlling motion of a surgical robot arm, the method comprising:
detecting a clash in respect of the surgical robot arm;
in response to detecting the clash, placing a limitation on the motion of the robot arm; and
receiving an input signal from a surgeon input device for controlling the surgical robot arm and sending an output signal to the surgical robot arm so as to control motion of the surgical robot arm in accordance with the limitation.

26. A non-transitory computer readable storage medium having stored thereon computer readable instructions that, when executed at a computer system, cause the computer system to perform the method of claim 25.
